(19) United States
(12) Patent Application Publication
(10) Pub. No.: US 2023/0168240 A1
(43) Pub. Date: Jun. 1, 2023

(54) SYSTEMS AND METHODS FOR HEMATOCRIT IMPEDANCE MEASUREMENT USING 4-WIRE MEASUREMENT

(71) Applicant: Trividia Health, Inc., Fort Lauderdale, FL (US)

(72) Inventors: Brent E. Modzelewski, Boca Raton, FL (US); Steven V. Leone, Lake Worth, FL (US)

(73) Assignee: Trividia Health, Inc., Fort Lauderdale, FL (US)

(21) Appl. No.: 18/072,496

(22) Filed: Nov. 30, 2022

Related U.S. Application Data
(60) Provisional application No. 63/284,295, filed on Nov. 30, 2021.

(51) Int. Cl. G01N 33/49; B01L 3/00

(52) U.S. Cl. CPC G01N 33/492; B01L 3/502715; B01L 2300/0645; B01L 2300/0825

(57) ABSTRACT

The present disclosure provides systems and methods for measuring a property of a sample. In some embodiments, the system comprises a test strip for collecting the sample, and a diagnostic measuring device configured to receive the test strip and measure a concentration of an analyte in the sample received on the test strip. The diagnostic measuring device further comprises a processor programmed to execute an analyte correction for correcting a measurement of the sample due to one or more interferents, including hematocrit, by using a four-wire sense circuit to measure HCT true complex impedance. The complex HCT impedance is mapped to HCT percentage by empirical methods and can then be used to compensate the glucose measurement for a more accurate blood glucose result.

### RELATED APPLICATIONS

This application claims the benefit of and priority to U.S. Provisional Application Serial No. 63/284,295, filed on Nov. 30, 2021, which is incorporated herein by reference in its entirety.

### FIELD

The present disclosure relates to systems and methods for hematocrit impedance measurement in connection with blood glucose and hemoglobin meters.

### BACKGROUND

Many industries have a commercial need to monitor the concentration of particular constituents in a fluid. In the health care field, for example, individuals with diabetes have a need to monitor a particular constituent within their bodily fluids. A number of systems are available that allow people to test a body fluid, such as, blood, urine, or saliva, to conveniently monitor the level of a particular fluid constituent, such as, cholesterol, proteins, and glucose. Such systems typically include a test strip where the user applies a fluid sample and a meter that “reads” the test strip to determine the level of the tested constituent in the fluid sample. A Blood Glucose Monitor (BGM) is an example of such a device. A hemoglobin meter (HbM) is another.

Conventionally, a BGM is a portable handheld device used to measure blood glucose levels for users with Type I or Type II diabetes. Typically, the user purchases small strips (approximately 20-30 mm x 5-9 mm) that interface with the BGM or HbM. The user draws a tiny amount of blood (fractional microliters) from a finger or other area using a lancer, applies a blood droplet sample onto the exposed end of the strip, and then inserts the connector end of the strip into the BGM connector port. A chemical reaction occurs between the blood sample and the chemistry on the strip, which is measured by the BGM to determine the blood glucose level in units of mg/dL or mmol/L, or kg/L depending on regional preferences. Units for hemobolgin (Hb) are in g/dL.

An issue that arises in measuring blood glucose level relates to interferents in the blood, such as hematocrit (HCT), that can affect the accuracy of the blood glucose measurement. Improved methods of measuring for interferents such as the HCT when using a blood glucose meter can be used to provide more accurate blood glucose readings to patients.

### SUMMARY

The present disclosure relates to systems and methods for hematocrit impedance measurement in connection with blood glucose and hemoglobin meters.

In some embodiments, a system for diagnostic testing is provided and includes a test strip and and an electronic meter for performing a diagnostic test on a sample applied to the test strip inserted therein. The electronic meter includes a housing having a test port for receiving the test strip, and a circuit configured to measure hematocrit. The circuit includes a first excitation contact configured for electrical communication with a first excitation trace on the test strip for sending an excitation signal to a HCT cathode on the test strip, a first sense contact configured for electrical communication with a first sense trace on the test strip for sending a sense measurement signal to the HCT cathode on the test strip, a second excitation contact configured for electrical communication with a second excitation trace on the test strip for sending an excitation signals to a HCT anode on the test strip, and a second sense contact configured for electrical communication with a second sensing trace on the test strip to sending a measurement signal to the HCT anode on the test strip. A processor can be programmed to perform the steps of measuring hematocrit in the sample placed onto the test strip using the sense measurement signals to calculate a hematocrit complex impedance value and mapping the calculated hematocrit impedance to a hematocrit concentration in the sample.

In some embodiments, the system further includes at least one glucose contact configured for electrical communication with at least one glucose trace on the test strip. In some embodiments, the processor further performs the step of calculating a concentration of glucose in the sample using the mapped hematocrit concentration and a glucose measurement from the at least one glucose trace.

In some embodiments, the system further includes a first glucose contact configured for electrical communication with a first glucose trace on the test strip and the HCT cathode on the test strip, and a second glucose contact configured for electrical communication with a second glucose trace on the test strip and the HCT anode.

In some embodiments, the system further includes at least one fill contact configured for electrical communication with at least one fill trace on the test strip.

In some embodiments, a system for diagnostic testing is provided and includes a test strip including first and second excitation traces for excitation signals and first and second sensing traces for separate sense measurement signals, and one or more electrodes in electrical communications with the first and second excitation traces and the first and second sense traces. The system also includes an electronic meter for performing a diagnostic test on a sample applied to the test strip inserted therein. The electronic meter includes a housing having a test port for receiving the test strip, a circuit configured to measure hematocrit and in electrical communication with the first and second excitation traces and the first and second sensing traces, and a processor programmed to perform the steps of measuring hematocrit in the sample placed onto the test strip using the sense measurement signals to calculate a hematocrit complex impedance value and mapping the calculated hematocrit impedance to a hematocrit concentration in the sample.

In some embodiments, the first excitation trace on the test strip is configured to send an excitation signal to a HCT cathode on the test strip, and the first sense trace on the test strip is configured to send a sense measurement signal to the HCT cathode on the test strip. In some embodiments, the second excitation trace on the test strip is configured to send an excitation signals to a HCT anode on the test strip, and the second sensing trace on the test strip is configured to send a measurement signal to the HCT anode on the test strip.

In some embodiments, the system further includes at least one glucose contact configured for electrical communication with at least one glucose trace on the test strip. In some embodiments, the processor further performs the step of calculating a concentration of glucose in the sample using the mapped hematocrit concentration and a glucose measurement from the at least one glucose trace.

In some embodiments, the system further includes at least one fill contact configured for electrical communication with at least one fill trace on the test strip.

In some embodiments, the a test strip is provided that includes a conductive pattern formed on a substrate including at least first and second electrode disposed on a proximal region of the substrate, first and second excitation electrical strip contacts and first and second sense electrical strip contacts disposed on a distal region of the substrate, a first excitation conductive trace and a first sense conductive trace electrically connecting the first electrode to the first excitation electrical strip contact and the first sense electrical strip contact, and a second excitation conductive trace and a second sense conductive trace electrically connecting the second electrode to the second excitation electrical strip contact and the second sense electrical strip contact; and a reagent layer contacting at least a portion of at least one electrode; wherein the at least first and second electrodes are in electrical communication with first and second contacts of a diagnostic meter configured to calculate a hematocrit concentration calculated from a measurement signals on the first and second sense conductive traces.

In some embodiments, the substrate is an electrically insulating layer.

In some embodiments, the test strip further includes at least one glucose electrode and at least one glucose electrical strip contact formed on the substrate. In some embodiments, the test strip further includes at least one glucose conductive trace configured to electrically connect the at least one glucose electrode to the at least one glucose electrical strip contact. In some embodiments, the test strip further includes at least one fill contact configured for electrical communication with at least one fill trace on the test strip.

In some embodiments, a method of determining hematocrit (HCT) concentration in a sample is provided and includes providing a sample on a test strip, the test strip including a conductive pattern formed on a substrate including at least first and second electrode disposed on a proximal region of the substrate, first and second excitation electrical strip contacts and first and second sense electrical strip contacts disposed on a distal region of the substrate, a first excitation conductive trace and a first sense conductive trace electrically connecting the first electrode to the first excitation electrical strip contact and the first sense electrical strip contact, and a second excitation conductive trace and a second sense conductive trace electrically connecting the second electrode to the second excitation electrical strip contact and the second sense electrical strip contact; measuring hematocrit in the sample on the test strip to calculate a hematocrit complex impedance value, and mapping the calculated hematocrit impedance to a hematocrit concentration in the sample.

In some embodiments, the method further includes applying an excitation signal to the sample such that a response to the excitation signal is analyzed to determine a glucose concentration in the blood sample. In some embodiments, the method further includes adjusting the glucose concentration in the blood sampled using the hematocrit concentration.

In some embodiments, the present disclosure provides a system for measuring a property of a sample comprising: a test strip for collecting the sample; a diagnostic measuring device configured to receive the test strip and measure a concentration of an analyte in the sample received on the test strip; and the diagnostic measuring device further comprising a processor programmed to execute an analyte correction for correcting a measurement of the sample due to one or more interferents, comprising: including hematocrit, by using a four-wire sense circuit to measure HCT true complex impedance. The complex impedance consists of a real and imaginary part. The real part being the resistive part and the imaginary part being the reactive part. The complex impedance can also be represented as a magnitude (in Ohms) and a phase (in degrees). The complex HCT impedance is mapped to HCT percentage by empirical methods and can then be used to compensate the glucose measurement for a more accurate blood glucose result.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1A is a general cross-sectional view of a test strip according to some embodiments of the present disclosure;

FIG. 1B is a top view of a conductive pattern on a substrate of a test strip according to some embodiments of the present disclosure;

FIGS. 2A and 2B illustrate a meter according to some embodiments of the present disclosure;

FIG. 3 illustrates an exemplary embodiment of a circuit to measure HCT for correcting glucose measurements; and

FIG. 4 is an exemplary flow chart showing an algorithm for correcting glucose measurements.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

In order to determine a measurement of an analyte, such as blood glucose, in a sample, such as blood, using a device, such as a blood glucose meter, certain interferents can be accounted for to increase the accuracy of the measurement. For example, one such interferent is the hematocrit (HCT) concentration in the blood.

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments.

Specific details are given in the following description to provide a thorough understanding of the embodiments. However, it will be understood by one of ordinary skill in the art that the embodiments may be practiced without these specific details. For example, systems, processes, and other elements in the presently disclosed embodiments may be shown as components in block diagram form in order not to obscure the embodiments in unnecessary detail. In other instances, well-known processes, structures, and techniques may be shown without unnecessary detail to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

Subject matter will now be described more fully with reference to the accompanying drawings, which form a part hereof, and which show, by way of illustration, specific example aspects and embodiments of the present disclosure. Subject matter may, however, be embodied in a variety of different forms and, therefore, covered or claimed subject matter is intended to be construed as not being limited to any example embodiments set forth herein; example embodiments are provided merely to be illustrative. The following detailed description is, therefore, not intended to be taken in a limiting sense.

A Blood Glucose Meter (BGM) is a portable, handheld device used to measure blood glucose levels for users with Type I or Type II diabetes. A Hemoglobin (HbG) meter measure blood hematocrit to compute Hemoglobin.

Typically, the user purchases tiny strips that interface with the BGM. The user draws a tiny amount of blood (a few microliters or less) from a finger or other area using a lancer. They then insert the strip into the BGM connector port. Now they apply the blood droplet onto the exposed end of the strip which has an open port for the blood. A chemical reaction occurs between the blood sample and the chemistry on the strip, which is measured by the BGM to determine the blood glucose level in units of mg/dL or mmol/L, depending on regional preferences and hematocrit as a percentage. Alternately, continuous blood glucose meters measure blood that is continuously provided via a patch. Hemoglobin is measured in g/dL.

The BGM measures blood glucose by analyzing the electrical response to an excitation signal. However, this response is dependent on the hematocrit (HCT) concentration in the blood. The accuracy of the glucose measurement is therefore dependent on the accuracy of the HCT concentration to compensate the measurement for this interferent.

The present disclosure provides systems and methods for hematocrit measurement. In particular, the present disclosure provides systems and methods for obtaining a hematocrit impedance measurement for a blood glucose meter.

A meter for measuring blood glucose or another analyst can include a portable, handheld device used to measure blood glucose levels for users with Type I or Type II diabetes. Typically, the user purchases test strips that interface with the meter. The user draws a tiny amount of blood (a few microliters or less) from a finger or other area using a lancer and a blood droplet is applied onto the exposed end of the strip which has an open port for the blood. The strip is inserted into the meter connector port and a chemical reaction occurs between the blood sample and the chemistry on the strip, which is measured by the meter to determine the blood glucose level in units of mg/dL or mmol/L, depending on regional preferences.

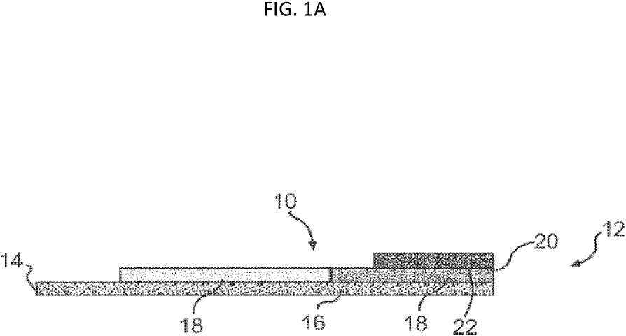

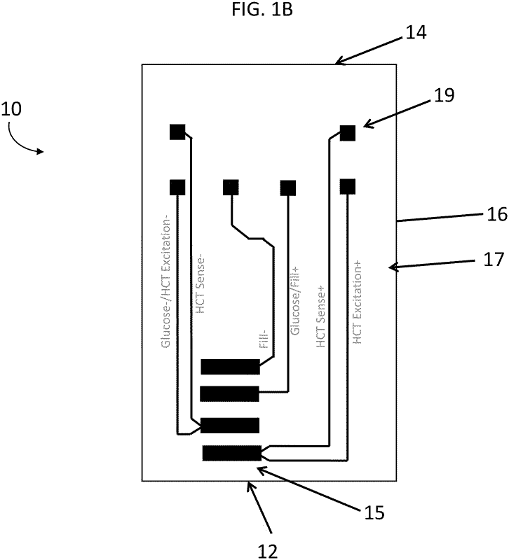

FIG. 1A illustrates a general cross-sectional view of an example embodiment of a test strip 10. In particular, FIG. 1A depicts a test strip 10 that includes a proximal end 12, a distal end 14, and is formed with a base layer 16 extending along the entire length of test strip 10. The base layer 16 can be composed of an electrically insulating material and has a thickness sufficient to provide structural support to test strip 10. For purposes of this disclosure, “distal” refers to the portion of a test strip further from the fluid source (e.g., closer to the meter) during normal use, and “proximal” refers to the portion closer to the fluid source (e.g., a fingertip with a drop of blood for a glucose test strip) during normal use. The base layer 16 may be composed of an electrically insulating material and has a thickness sufficient to provide structural support to test strip 10.

As seen in FIG. 1A, the proximal end 12 of test strip 10 includes a sample receiving location, such as a sample chamber 20 configured to receive a patient’s fluid sample, as described above. The sample chamber 20 may be formed in part through a slot in a dielectric insulating layer 18 formed between a cover 22 and the underlying measuring electrodes formed on the base layer 16. Accordingly, the sample chamber 20 may include a first opening, e.g., a sample receiving location, in the proximal end of the test strip and a second opening for venting the sample chamber 20. The sample chamber 20 may be dimensioned to be able to draw the blood sample in through the first opening, and to hold the blood sample in the sample chamber 20, by capillary action. The test strip 10 can include a tapered section that is narrowest at the proximal end 12 or can include other indicia to make it easier for the user to locate the first opening and apply the blood sample.

In reference to FIG. 1B, in accordance with an example embodiment of the present disclosure, the strip 10 can include a conductive pattern disposed on base layer 16 of the strip 10. In some embodiments, the conductive pattern may be formed by laser ablating the electrically insulating material of the base layer 16 to expose the electrically conductive material underneath. Other methods may also be used, such as inserted conductors with physical attachment to control circuit. Other methods may also be used to dispose the conductive pattern on the base layer. The conductive pattern may include a plurality of electrodes 15 disposed on base layer 16 near proximal end 12, a plurality of electrical strip contacts 19 disposed on base layer 16 near distal end 14, and a plurality of conductive traces 17 electrically connecting the electrodes 15 to the plurality of electrical strip contacts 19.

In some embodiments, a reagent layer may be disposed on the base layer 16 of the strip 10 in contact with at least a working electrode of the conductive pattern. The reagent layer may include an enzyme, such as glucose oxidase, and a mediator, such as potassium ferricyanide or ruthenium hexamine. Reagent layer 90 may also include other components, such as buffering materials (e.g., potassium phosphate), polymeric binders (e.g., hydroxypropyl-methyl-cellulose, sodium alginate, microcrystalline cellulose, polyethylene oxide, hydroxyethylcellulose, and/or polyvinyl alcohol), and surfactants (e.g., Triton X-100 or Surfynol 485). With these chemical constituents, the reagent layer reacts with glucose in the blood sample in the following way. The glucose oxidase initiates a reaction that oxidizes the glucose to gluconic acid and reduces the ferricyanide to ferrocyanide. When an appropriate voltage is applied to working electrode, relative to counter electrode, the ferrocyanide is oxidized to ferricyanide, thereby generating a current that is related to the glucose concentration in the blood sample. As would be appreciated by one skilled in the art, any combination of strips 10 known in the art can be utilized without departing from the scope of the present disclosure.

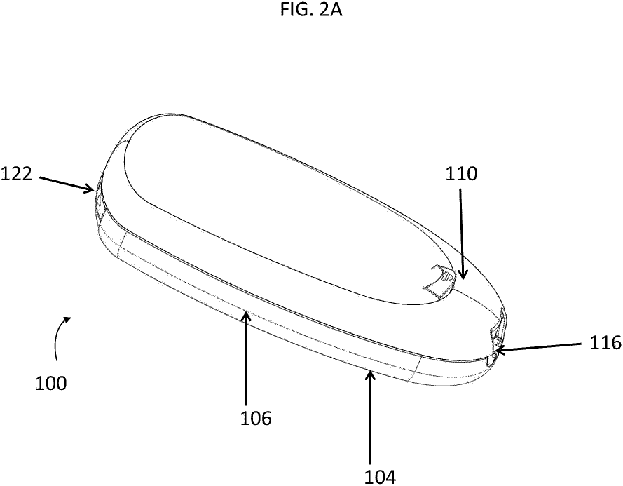

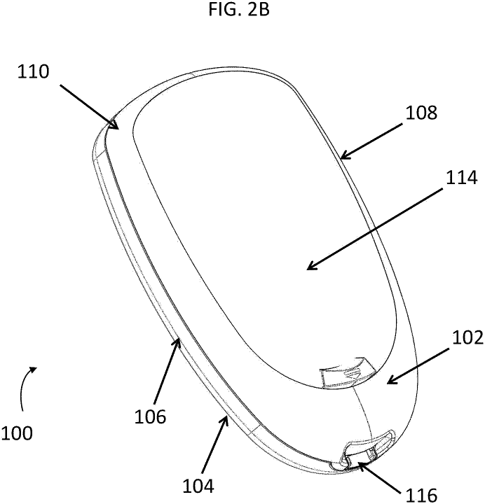

FIG. 2A and FIG. 2B illustrate an exemplary illustration of a meter 100 used to measure the glucose level in a blood sample. The meter 100 includes a housing having a test port for receiving the test strip, and a processor or microprocessor programmed to perform methods and algorithms to determine glucose concentration in a test sample or control solution as disclosed in the present disclosure. In some embodiments, the meter 100 has a size and shape to allow it to be conveniently held in a user’s hand while the user is performing the glucose measurement. The meter 100 may include a front side 102, a back side 104, a left side 106, a right side 108, a top side 110, and a bottom side 112. The front side 102 may include a display 114, such as a liquid crystal display (LCD). A bottom side 112 may include a strip connector 116 into which test strip can be inserted to conduct a measurement. The meter 100 may also include a storage device for storing test algorithms or test data. The top side 110 may include one or more user controls 122, such as buttons, with which the user may control meter 100, and the right side 108 may include a serial connector (not shown).

In some embodiments, the blood glucose meter comprises a decoder for decoding a predetermined electrical property, e.g. resistance, from the test strips as information. The decoder operates with, or is a part of, the microprocessor.

The meter can be programmed to wait for a predetermined period of time after initially detecting the blood sample, to allow the blood sample to react with the reagent layer or can immediately begin taking readings in sequence. During a fluid measurement period, the meter applies an assay voltage between the working and counter electrodes and takes one or more measurements of the resulting current flowing between the working and counter electrodes. The assay voltage is near the redox potential of the chemistry in the reagent layer, and the resulting current is related to the concentration of the particular constituent measured, such as, for example, the glucose level in a blood sample.

In one example, the reagent layer may react with glucose in the blood sample to determine the particular glucose concentration. In one example, glucose oxidase is used in the reagent layer. The recitation of glucose oxidase is intended as an example only and other materials can be used without departing from the scope of the disclosure. Other possible mediators include, but are not limited to, ruthenium and osmium. During a sample test, the glucose oxidase initiates a reaction that oxidizes the glucose to gluconic acid and reduces the ferricyanide to ferrocyanide. When an appropriate voltage is applied to a working electrode, relative to a counter electrode, the ferrocyanide is oxidized to ferricyanide, thereby generating a current that is related to the glucose concentration in the blood sample. The meter then calculates the glucose level based on the measured current and on calibration data that the meter has been signaled to access by the code data read from the second plurality of electrical contacts associated with the test strip. The meter then displays the calculated glucose level to the user.

A correction based on a measured HCT value can be applied to glucose level determined by the meter. In some embodiments, the HCT measurement sequence begins after a drop of blood or control is detected when the drop completes the circuit between the HCT measurement anode and cathode. In some embodiments, the HCT is analyzed based on an electrical measurement between two electrodes on the test strip separate from the electrodes used to measure glucose, or the electrodes can be shared for both measurements. After the drop is detected on the HCT electrodes a Fill excitation tests for when the sample has completely filled the test strip. Then, either before, during, or after glucose measurement in the case of a glucose meter, an AC excitation voltage signal is applied to the HCT electrodes. The salt content of blood creates an electronic signature, in which the magnitude and phase response can be mapped to the HCT of the blood. The impedance of the electrical signature is affected by temperature, so the true HCT reading is corrected for temperature for the temperature difference from 24° C. (dT).

In some embodiments, the glucose measurement sequence is initiated only when the meter detects a full sample chamber. The glucose in the test sample is oxidized by the enzyme glucose dehydrogenase-FAD, producing gluconolactone and the reduced form of an electron mediator. The reduced mediator is then oxidized at the surface of the glucose measurement anode to produce an electrical signal (current in nanoamp units) that is detected by the meter. The electrical signal (current, in nanoamps) produced by oxidation of the reduced mediator at the surface of the glucose measurement anode is proportional to the amount of glucose in the test sample. The HCT value (which can be temperature corrected) is then used to determine the temperature corrected glucose value.

The meter can measure blood glucose by analysing the electrical response to an excitation signal. However, this response is dependent on the HCT concentration in the blood. The accuracy of the glucose measurement is therefore dependent on the accuracy of the HCT concentration to compensate the measurement for this interferent. For a given blood glucose sample, the peak response current to a voltage excitation used to measure blood glucose on the blood sample can be inversely proportional to the HCT concentration in the blood. Knowing the HCT impedance provides the data to map the HCT concentration to the peak current through empirical methods. This known HCT concentration (% HCT) can then be used to adjust blood glucose measurement. Hemoglobin concentration is converted directly from percent HCT.

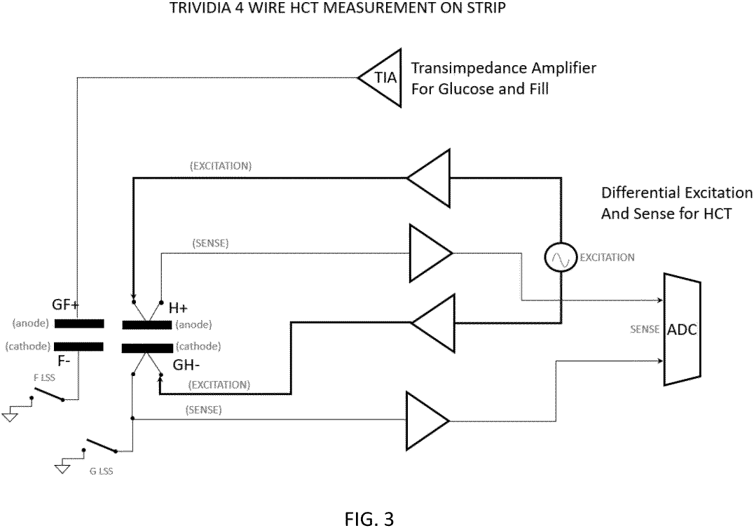

Various methods exist for measuring the HCT concentration from step response to impedance measurement. In some embodiments, a 4-wire HCT measurement design can be used, as shown in FIG. 3. Four wire measurements can be used such that measurements for sensing and excitation signals are on separate wires/traces. When sense measurements are performed on the same traces as excitation signals, voltage drops and phase shifts associated with the shared traces cannot be separated or accounted for, and the measurement is performed at the IC pin. When using a four-wire HCT measurement design, the measurement is performed at the reaction well. As shown, the HCT is driven differentially with an AC voltage and the HCT sample is measured at the reaction well on the strip with a differential sensing circuit to the ADC. In this way, the trace lines, connector and strip characteristics can be eliminated from the HCT measurement. Using four-wire sensing on the HCT compensates for signal parasitic parameters in the PCB (Printed Circuit Board), strip connector, and electrode traces on the strip. This is realized by bypassing the excitation traces and measuring with two independent traces that have zero voltage drop or phase shift. Such parasitic parameters include stray resistance, capacitance, and inductance. Since the sense electrodes tie directly across the HCT electrodes, they bypass these parasitic parameters for a more accurate HCT measurement. As shown, there are two separate excitation signals and two separate sense (measurement) signals to achieve the four wire configuration.

Also shown in FIG. 3 is the TIA to measure the DC current for a DC excitation on Glucose and Fill with the associated low side switch (LSS). The TIA (Transimpedance Amplifier) converts current to voltage for conversion in an analog to digital converter on the microcontroller.

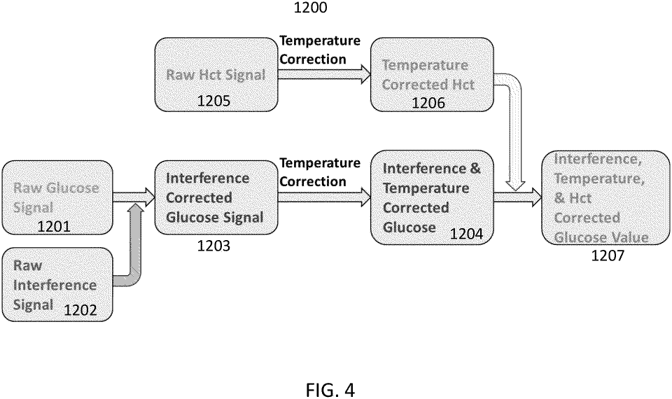

In some embodiments, the systems of the present disclosure may be used to measure glucose concentration in blood, among other measurements, as discussed above. Once the meter has performed an initial check routine, the meter can apply a drop-detect voltage between working and counter electrodes and detect a fluid sample, for example, a blood sample, by detecting a current flow between the working and counter electrodes (i.e., a current flow through the blood sample as it bridges the working and counter electrodes). For example, in some embodiments, the meter may measure an amount of components in blood which may impact the glucose measurement, such as, for example, a level of hematocrit or of an interferant. The meter can later use such information to adjust the glucose concentration to account for the hematocrit level and the presence of the interferants in blood, among other things. These measurements can also be corrected based on the temperature. The meter can then adjust the glucose level, as necessary, based on the measurements of the temperature, hematocrit and the presence of interferants. Non-limiting examples of algorithms for glucose level correction are presented in FIG. 4. Errors can be displayed if encountered.

FIG. 4 is an embodiment flow chart for correcting the analyte value 1200, wherein the analyte specific current is modified based on temperature and hematocrit and interference currents to then generate a corrected analyte value. For example, equations may be IC = IA - S×II, where IC is the corrected current, IA is the current measured from the analyte anode, II is the current measured from the interference anode, and S is an empirically derived scaling factor. The present calculation may eliminate the need to make complicated calculation and/or voltage application schemes. The present calculation uses a mathematically modified (scaled) subtraction of the interference current from the current from the analyte specific anode. The interference current may be multiplied by an empirically determined constant that is dependent only on the relative areas of the two electrodes, not on the relative effects of hematocrit and temperature variations on the two currents. This is because the two reagents (analyte and interference) are formulated to respond the same way to hematocrit and temperature variations. Thus, referring to FIG. 4, the raw glucose signal 1201 would be corrected with the raw interference signal 1202 to obtain an interference corrected glucose signal 1203, where a temperature correction is incorporated to obtain an interference and temperature corrected glucose value 1204. The raw Hct signal 1205 is corrected to obtain a temperature corrected Hct 1206. The interference & temperature corrected glucose value 1204 may then be incorporated with the temperature corrected Hct 1206 to obtain an interference, temperature & Hct corrected glucose value 1207.

It is also possible to first make temperature and hematocrit adjustments to the interference current and then subtract it from the raw analyte current and then subject that corrected current to another temperature and hematocrit adjustment. In some embodiments, it may be possible to correct the analyte and interference currents separately for temperature and hematocrit, and then convert each separately to an uncorrected glucose value and to a glucose equivalent value, respectively. Then the glucose equivalent value can be subtracted from the uncorrected glucose value to obtain a corrected glucose value.

In some embodiments, it is possible to use the present calculation to also first convert the interference current to analyte equivalents and then subtract it from the amount of analyte of interference and subtract that number. That is, the correction can occur before or after mathematically processing the current. For example, by having the interference anode larger for improved signal to noise ratio due to the currents being so small, at least one aspect includes using a scaling factor and anodes of different surface area.

It should be noted that while the operation of the system of the present disclosure has been described primarily in connection with determining glucose concentration in blood, the systems of the present disclosure may be configured to measure other analytes in blood as well as in other fluids, as discussed above.

The meter can include one or more processors configured to execute instructions encoded on at least one non-transitory computer-readable storage medium. Execution of the instructions encoded on the at least one non-transitory computer-readable storage medium can cause the one or more processors to carry out one or more above the above-described methods.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the disclosure. It is intended that the present disclosure be limited only to the extent required by the appended claims and the applicable rules of law.

It is also to be understood that the following claims are to cover all generic and specific features of the disclosure described herein, and all statements of the scope of the disclosure which, as a matter of language, might be said to fall therebetween.

What is claimed is:

1. A system for diagnostic testing, comprising:
a test strip; and
an electronic meter for performing a diagnostic test on a sample applied to the test strip inserted therein, the electronic meter comprising
a housing having a test port for receiving the test strip,
a circuit configured to measure hematocrit, the circuit comprising:
a first excitation contact configured for electrical communication with a first excitation trace on the test strip for sending an excitation signal to a HCT cathode on the test strip,
a first sense contact configured for electrical communication with a first sense trace on the test strip for sending a sense measurement signal to the HCT cathode on the test strip,
a second excitation contact configured for electrical communication with a second excitation trace on the test strip for sending an excitation signals to a HCT anode on the test strip,
a second sense contact configured for electrical communication with a second sensing trace on the test strip to sending a measurement signal to the HCT anode on the test strip, and
a processor programmed to perform the steps of:
measuring hematocrit in the sample placed onto the test strip using the sense measurement signals to calculate a hematocrit complex impedance value; and
mapping the calculated hematocrit impedance to a hematocrit concentration in the sample.

2. The system of claim 1, further comprising at least one glucose contact configured for electrical communication with at least one glucose trace on the test strip.

3. The system of claim 2, wherein the processor further performs the step of calculating a concentration of glucose in the sample using the mapped hematocrit concentration and a glucose measurement from the at least one glucose trace.

4. The system of claim 1, further comprising a first glucose contact configured for electrical communication with a first glucose trace on the test strip and the HCT cathode on the test strip, and a second glucose contact configured for electrical communication with a second glucose trace on the test strip and the HCT anode.

5. The system of claim 1, further comprising at least one fill contact configured for electrical communication with at least one fill trace on the test strip.

6. A system for diagnostic testing, comprising:
a test strip comprising
first and second excitation traces for excitation signals and first and second sensing traces for separate sense measurement signals; and
one or more electrodes in electrical communications with the first and second excitation traces and the first and second sense traces; and
an electronic meter for performing a diagnostic test on a sample applied to the test strip inserted therein, the electronic meter comprising:
a housing having a test port for receiving the test strip,
a circuit configured to measure hematocrit and in electrical communication with the first and second excitation traces and the first and second sensing traces, and
a processor programmed to perform the steps of:
measuring hematocrit in the sample placed onto the test strip using the sense measurement signals to calculate a hematocrit complex impedance value; and
mapping the calculated hematocrit impedance to a hematocrit concentration in the sample.

7. The system of claim 6, wherein the first excitation trace on the test strip is configured to send an excitation signal to a HCT cathode on the test strip, and the first sense trace on the test strip is configured to send a sense measurement signal to the HCT cathode on the test strip.

8. The system of claim 6, wherein the second excitation trace on the test strip is configured to send an excitation signals to a HCT anode on the test strip, and the second sensing trace on the test strip is configured to send a measurement signal to the HCT anode on the test strip.

9. The system of claim 6, further comprising at least one glucose contact configured for electrical communication with at least one glucose trace on the test strip.

10. The system of claim 9, wherein the processor further performs the step of calculating a concentration of glucose in the sample using the mapped hematocrit concentration and a glucose measurement from the at least one glucose trace.

11. The system of claim 6, further comprising at least one fill contact configured for electrical communication with at least one fill trace on the test strip.

12. A test strip, comprising:
a conductive pattern formed on a substrate comprising at least first and second electrode disposed on a proximal region of the substrate, first and second excitation electrical strip contacts and first and second sense electrical strip contacts disposed on a distal region of the substrate, a first excitation conductive trace and a first sense conductive trace electrically connecting the first electrode to the first excitation electrical strip contact and the first sense electrical strip contact, and a second excitation conductive trace and a second sense conductive trace electrically connecting the second electrode to the second excitation electrical strip contact and the second sense electrical strip contact; and
a reagent layer contacting at least a portion of at least one electrode;
wherein the at least first and second electrodes are in electrical communication with first and second contacts of a diagnostic meter configured to calculate a hematocrit concentration calculated from a measurement signals on the first and second sense conductive traces.

13. The test strip of claim 12, where in the substrate is an electrically insulating layer.

14. The test strip of claim 12, further comprising at least one glucose electrode and at least one glucose electrical strip contact formed on the substrate.

15. The test strip of claim 14, further comprising at least one glucose conductive trace configured to electrically connect the at least one glucose electrode to the at least one glucose electrical strip contact.

16. The test strip of claim 12, further comprising at least one fill contact configured for electrical communication with at least one fill trace on the test strip.

17. A method of determining hematocrit (HCT) concentration in a sample, comprising:
providing a sample on a test strip, the test strip comprising a conductive pattern formed on a substrate comprising at least first and second electrode disposed on a proximal region of the substrate, first and second excitation electrical strip contacts and first and second sense electrical strip contacts disposed on a distal region of the substrate, a first excitation conductive trace and a first sense conductive trace electrically connecting the first electrode to the first excitation electrical strip contact and the first sense electrical strip contact, and a second excitation conductive trace and a second sense conductive trace electrically connecting the second electrode to the second excitation electrical strip contact and the second sense electrical strip contact;
measuring hematocrit in the sample on the test strip to calculate a hematocrit complex impedance value; and
mapping the calculated hematocrit impedance to a hematocrit concentration in the sample.

18. The method of claim 17, further comprising applying an excitation signal to the sample such that a response to the excitation signal is analyzed to determine a glucose concentration in the blood sample.

19. The method of claim 18, further comprising adjusting the glucose concentration in the blood sampled using the hematocrit concentration.
